(19) United States
(12) Patent Application Publication
(10) Pub. No.: US 2005/0155611 A1
(43) Pub. Date: Jul. 21, 2005

(54) Surgical sealing device

(76) Inventors: Trevor Vaugh, Birr (IE); Frank Bonadio, Bray (IE); John Butler, Blackrock (IE)

Correspondence Address: Finnegan, Henderson, Farabow,;Garrett & Dunner, L.L.P., 1300 I Street, N.W., Washington, DC, 20005-3315, US

(21) Appl. No.: 10/981,730

(22) Filed: Nov. 5, 2004

Related U.S. Application Data
(60) Provisional application No. 60/517,102, filed on Nov. 5, 2003.
(60) Provisional application No. 60/535,807, filed on Jan. 13, 2004.
(60) Provisional application No. 60/566,186, filed on Apr. 29, 2004.

(51) Int. Cl. A61M 5/14; A61B 17/00

(52) U.S. Cl. 128/887

(57) ABSTRACT

A surgical sealing device comprises sealing device of a gelatinous elastomeric material, a distal ring element and a proximal ring element. The ring elements are twisted relative to one another to twist the valve body into a twisted configuration to seal across a wound opening. The ring elements are snap-fitted together to maintain the valve in the twisted configuration. By passing an object, such as surgeon's hand through the valve, the opposing sidewalls of a lumen through the valve may be forced radially apart to partially open the lumen, and thereby enable the surgeon's hand to access the interior of the abdomen. The valve remains twisted during insertion of the surgeon's hand through the valve to minimise the loss of insufflation gases from the abdomen.

This invention relates to a surgical sealing device suitable for use during a surgical procedure. In particular the invention relates to a surgical sealing device suitable for sealing around an object, such as a surgeon's hand and/or forearm or a surgical instrument, while facilitating access to an internal cavity.

In carrying out a surgical procedure in the region of the abdomen it is known to form an incision and then retract sides of the incision to provide an access opening to the interior of the abdomen. The opening may be sealed with a valve in an effort to prevent insufflation gases from escaping and to maintain pneumoperitoneum. It is however difficult to provide an effective gas seal while providing access for an object, such as a surgeon's hand and/or forearm or a surgical instrument.

This invention is directed towards providing a surgical sealing device which will address these problems.

### STATEMENTS OF INVENTION

According to the invention there is provided a surgical sealing device comprising a sealing valve for sealing across an opening to an internal cavity, the sealing valve having a material which is twisted to form a seal, the seal being maintained upon passage of an object therethrough.

In another aspect of the invention there is provided a surgical sealing device comprising a sealing valve for sealing across an opening to an internal cavity;
the sealing valve having a material defining a lumen;
the material being twisted to close the lumen;
the lumen being at least partially opened upon passage of an object through the lumen.

In another aspect of the invention there is provided a surgical sealing device comprising a sealing valve for sealing across an opening to an internal cavity:
the sealing valve having a lumen extending therethrough;
the valve being twisted to close the lumen for sealing across the opening;
the valve being movable substantially in a radial direction to enable the lumen to be at least partially opened for passage of an object through the lumen to access the internal cavity, while the valve remains twisted.

In a further aspect of the invention provides a surgical sealing device comprising a sealing valve for sealing across an opening to internal cavity;
the sealing valve having a lumen extending therethrough;
the valve being twisted to close the lumen for sealing across the opening;
the lumen being at least partially openable upon engagement of an object with the closed lumen and passage of the object through the lumen to access the internal cavity, while the valve remains twisted.

The invention also provides in another aspect a surgical sealing device comprising a sealing valve for sealing across an opening to an internal cavity;
the sealing valve having a lumen extending therethrough;
the valve being twisted to close the lumen for sealing across the opening;
the valve being configured to enable the lumen to be at least partially opened for passage of an object through the lumen to access the internal cavity, while the valve remains twisted;
the valve being constrained longitudinally.

In another aspect the invention provides a surgical sealing device comprising a sealing valve for sealing across an opening to an internal cavity;
the sealing valve having a lumen extending therethrough;
a first part of the valve being twistable relative to a second part of the valve between an untwisted configuration, in which the lumen is open, and a twisted configuration to close the lumen for sealing across the opening;
in the twisted configuration, the valve being configured to enable the lumen to be at least partially opened for passage of an object through the lumen to access the internal cavity.

In one embodiment of the invention the material defines a lumen extending therethrough, the lumen being closed when the material is twisted to form the seal. The material may be configured to enable the lumen to be at least partially opened for passage of an object through the lumen to access the internal cavity, while the lumen remains twisted.

In one embodiment the material sealingly engages the object upon passage therethrough. The lumen may be at least partially opened while the material remains twisted.

In one case a first part of the material is twistable relative to a second part of the material between an untwisted configuration and a twisted configuration. In the untwisted configuration, the lumen may be open. In the twisted configuration, the lumen may be closed.

In one case the valve is at least partially of a gelatinous elastomeric material.

The first part of the valve may comprise a distal end part of the valve. The second part of the valve may comprise a proximal end part of the valve.

In another case, in the twisted configuration, a third part of the valve is movable relative to a fourth part of the valve to at least partially open the lumen. The third part of the valve may be movable substantially radially relative to the fourth part of the valve. The third part of the valve may comprise a part of a sidewall of the lumen. The fourth part of the valve may comprise an opposing part of the sidewall of the lumen.

In another embodiment, in the twisted configuration, the valve is constrained longitudinally. In the twisted configuration, the location of the first part of the valve may be longitudinally fixed relative to the location of the second part of the valve. In the twisted configuration, the first part of the valve may engage the second part of the valve.

In the twisted configuration, the lumen may be at least partially openable upon engagement of an object with the closed lumen and passage of the object through the lumen.

In the twisted configuration, the valve may be configured to effect a seal between a sidewall of the lumen and an object inserted through the lumen.

In the twisted configuration, the lumen may be biased towards being closed. In one case the valve is of a resilient material to bias the lumen towards being closed.

In another embodiment the valve comprises a generally cylindrical sealing body which is twistable into the twisted configuration. The generally cylindrical sealing body may be formed by moulding or extrusion. The generally cylindrical sealing body may be formed from a generally flat sheet of material.

In one case the first part of the material and/or the second part of the material is turned radially outwardly. The first part of the material and/or the second part of the material may be provided in the form of a flange.

In one case the device comprises a lock for maintaining the valve in the twisted configuration. The lock may comprise a first lock part attached to the first part of the valve and a second lock part attached to the second part of the valve, the first lock part being coupleable to the second lock part to maintain the first part of the valve twisted relative to the second part of the valve. The first lock part and the second lock part may be configured to be snap-fitted together. In one case the lock part is attached to the part of the valve by overmoulding a part of the valve over at least part of the lock part. The lock part may comprise one or more overmould openings therein.

In another case the lock part is attached to the part of the valve by a mechanical attachment arrangement. The mechanical attachment arrangement may comprise one or more protruding male elements for co-operative reception in one or more female recesses. The lock part may comprise the one or more male elements and the part of the valve comprises the one or more female recesses.

The lock part may be rigid relative to the valve.

The lock part may comprise a ring element.

In another embodiment the device comprises a grip element for. gripping of the valve. The device may comprise a first grip element attached to the first part of the valve and a second grip element attached to the second part of the valve. In one case the grip element is attached to the part of the valve by overmoulding a part of the valve over at least part of the grip element. The grip element may comprise one or more overmould openings therein.

In another embodiment the grip element is attached to the part of the valve by a mechanical attachment arrangement. The mechanical attachment arrangement may comprise one or more protruding male elements for co-operative reception in one or more female recesses. The grip element may comprise the one or more male elements and the part of the valve comprises the one or more female recesses.

In one case the grip element is rigid relative to the valve.

In one case the grip element comprises a ring element.

In a further embodiment the device is mountable to a retractor device. The device may comprise a mounting element for mounting the device to a retractor device. The mounting element may be fixedly attached to the valve.

In one case a device as claimed in claim 50 wherein a part of the valve is overmoulded over at least part of the mounting element. The mounting element may comprise one or more overmould openings therein.

The mounting element may be attached to the valve by a mechanical attachment arrangement. The mechanical attachment arrangement may comprise one or more protruding male elements for co-operative reception in one or more female recesses. The mounting element may comprise the one or more male elements and the valve may comprise the one or more female recesses.

In one case the mounting element comprises an engagement formation for snap-fit mounting of the device to retractor device.

The mounting element may comprise a screw-thread formation for screw-thread mounting of the device to a retractor device.

In one case the mounting element is rigid relative to the valve. In one case mounting element comprises a ring element.

The device may be configured to effect a seal between the device and a retractor device upon mounting of the device to the retractor device. In one case at least part of the valve is configured to engage a retractor device upon mounting.

The mounting element may comprise a sleeve for extending between the valve and a retractor device. The sleeve may be hookable around a part of a retractor device to mount the device to the retractor device. The sleeve may comprise a mounting ring for hooking around a part of a retractor device. The mounting ring may be an O-ring. In one case a distal end of the sleeve is coupled to the valve and a proximal and of the sleeve is coupleable to a retractor device. The device may comprise a sealing element for sealingly engaging the mounting element to a retractor device. The sealing element may comprise a sealing ring.

The gelatinous elastomeric material may comprise a plasticiser. In one case the plasticiser is selected from the group consisting of naturally derived oils, synthetic oils and liquid oligomers. The gelatinous elastomeric material may be a silicone based gel.

In one case the valve is configured to be located externally of an internal cavity proximally of an opening to the internal cavity.

The valve may be configured to be located within an internal cavity distally of an opening to the internal cavity.

The invention also provides in another aspect a surgical assembly comprising:
a retractor device for retracting the sides of an opening to an internal cavity; and
a surgical sealing device of the invention.

In one case the surgical sealing device is mounted to the retractor device. The retractor device may have a distal end and a proximal end, and the surgical sealing device may be located adjacent to the distal end of the retractor device. The retractor device may have a distal end and a proximal end, and the surgical sealing device may be located adjacent to the proximal end of the retractor device.

In another aspect of the invention there is provided a method of accessing an internal cavity through an opening to the internal cavity, the method comprising the steps of:
providing a sealing valve, the valve being twisted to form a seal;
arranging the twisted valve to seal across the opening; and
passing an object through the twisted valve, while maintaining the seal, to access the internal cavity.

In one embodiment the valve has a lumen extending therethrough, and the valve is twisted into a twisted configuration to close the lumen and form the seal. The lumen may be at least partially opened by passing of the object through the twisted valve. The step of passing an object through the valve in the twisted configuration may cause a third part of the valve to move relative to a fourth part of the valve to at least partially open the lumen. The third part of the valve may be moved substantially radially relative to the fourth part of the valve.

In one case the method comprises the step of longitudinally constraining the valve in the twisted configuration. The method may comprise the step of twisting the valve from an untwisted configuration, in which the lumen is open, to the twisted configuration. The valve may be pre-twisted into the twisted configuration. The valve may be supplied in the twisted configuration. The step of twisting the valve may be performed during manufacture of the sealing valve. The step of twisting the valve may be performed during a surgical procedure.

In one case the method comprises the step of hooking the valve in the twisted configuration.

The valve may be arranged within the opening. The valve may be arranged externally of the internal cavity proximally of the opening. The valve may be arranged within the internal cavity distally of the opening.

In another embodiment the method comprises the step of effecting a seal between a sidewall of the lumen and the object passing through the lumen.

The method may comprise the step of retracting the sides of the opening to the internal cavity. In one case the method comprises the step of mounting the valve to a retractor device. The method may comprise the step of effecting a seal between the valve and the retractor device.

The object passed through the lumen may comprise a surgeon's hand and/or forearm.

The object passed through the opening may comprise a surgical instrument.

In a further aspect the invention provides a method of accessing an internal cavity through an opening to the internal cavity, the method comprising the steps of:
providing a sealing valve, having a lumen extending therethrough, the sealing valve being twisted into a twisted configuration to close the lumen closed;
arranging the valve in the twisted configuration to seal across the opening;
passing an object through the valve in the twisted configuration to at least partially open the lumen; and
passing the object through the lumen to access the internal cavity.

### BRIEF DESCRIPTION OF THE DRAWINGS

The invention will be more clearly understood from the following description of some embodiments thereof, given by way of example only, with reference to the accompanying drawings, in which:-

FIG. 1 is a perspective view of a sealing valve of a surgical sealing device according to the invention;

FIG. 2 is a cut-away, perspective view of a distal ring element and a proximal ring element of a surgical sealing device according to the invention;

FIG. 3 is a cut-away, perspective view of a surgical sealing device according to the invention in an untwisted configuration including the sealing valve of FIG. 1 and the ring element of FIG. 2;

FIG. 4 is a cross-sectional, front view of the device of FIG. 3 in the untwisted configuration;

FIGS. 5 and 6 are enlarged views of parts of the device of FIG. 4;

FIG. 7 is a cut-away, perspective view of a part of an extractor device;

FIG. 8 is a cross-sectional, front view of the device of FIG. 3 in a twisted configuration mounted to a retractor device;

FIG. 9 is a cut-away, perspective view of the ring elements of FIG. 2 mounted to the retractor device part of FIG. 7;

FIG. 10 is a cross-sectional, front view of the ring elements of FIG. 2 mounted to the retractor device part of FIG. 7;

FIG. 11 is a cross-sectional, front view of the device of FIG. 3 in the twisted configuration mounted to the retractor device of FIG. 8 with a surgeon's hand/forearm passing through the device;

FIG. 12 is a cut-away, perspective view of another surgical sealing device according to the invention in an untwisted configuration being mounted to a part of another retractor device;

FIG. 13 is a perspective view of a generally cylindrical sealing valve of another surgical sealing device according to the invention;

FIG. 14 is a perspective view of a ring element of another surgical sealing device according to the invention;

FIG. 15 is an exploded, perspective view of another surgical sealing device according to the invention in an untwisted configuration including the sealing valve of FIG. 13 and the ring element of FIG. 14;

FIG. 16 is a perspective view of the device of FIG. 15 in the untwisted configuration;

FIG. 17 is a perspective view of the device of FIG. 15 in a twisted configuration;

FIG. 18 is a perspective view of another surgical sealing device according to the invention in a twisted configuration;

FIG. 19 is a plan view from above of the device of FIG. 18 in the twisted configuration;

FIG. 20 is a perspective view of a cylindrical body used to form a sealing valve of the device of FIG. 18;

FIGS. 21 and 22 are perspective views illustrating an alternative method of forming a sealing valve of the device of FIG. 18;

FIG. 23 is a partially cross sectional, plan view from above of the device of FIG. 18 in the twisted configuration with a relatively large object, such as a surgeon's hand/forearm passing through the device;

FIG. 24 is a partially cross sectional, plan view from above of the device of FIG. 18 in the twisted configuration with a smaller object, such as an instrument passing through the device;

FIG. 25 is a cross sectional, front view of the device of FIG. 18 in the twisted configuration mounted to a retractor device;

FIG. 26 is a cross sectional, front view of the valve of FIG. 18 in the twisted configuration mounted to the retractor device of FIG. 25 with a surgeon's hand/forearm passing through the device;

FIG. 27 is a cross sectional, front view of another surgical sealing device according to the invention mounted to the retractor device of FIG. 25;

FIG. 28 is a cross sectional, front view of another surgical sealing device according to the invention mounted to the retractor device of FIG. 25; and

FIG. 29 is a cross sectional, front view of another surgical sealing device according to the invention mounted to the retractor device of FIG. 25;

### DETAILED DESCRIPTION

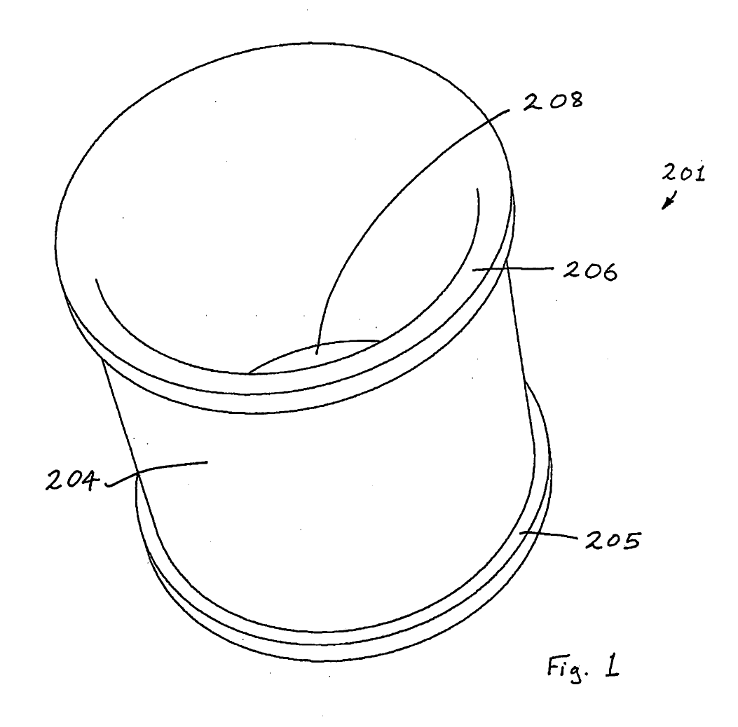

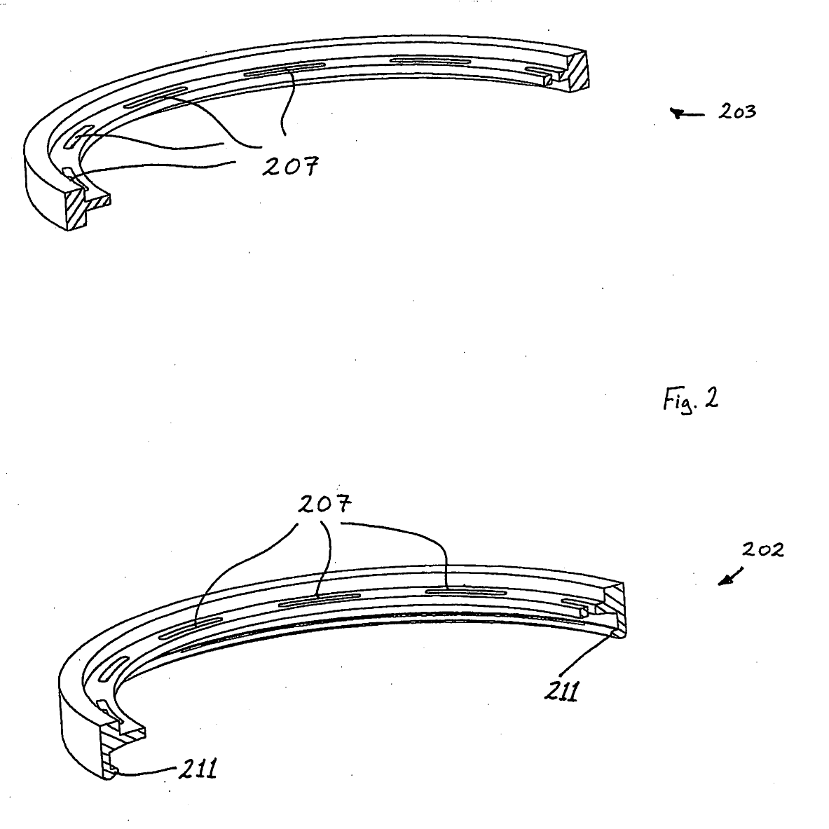

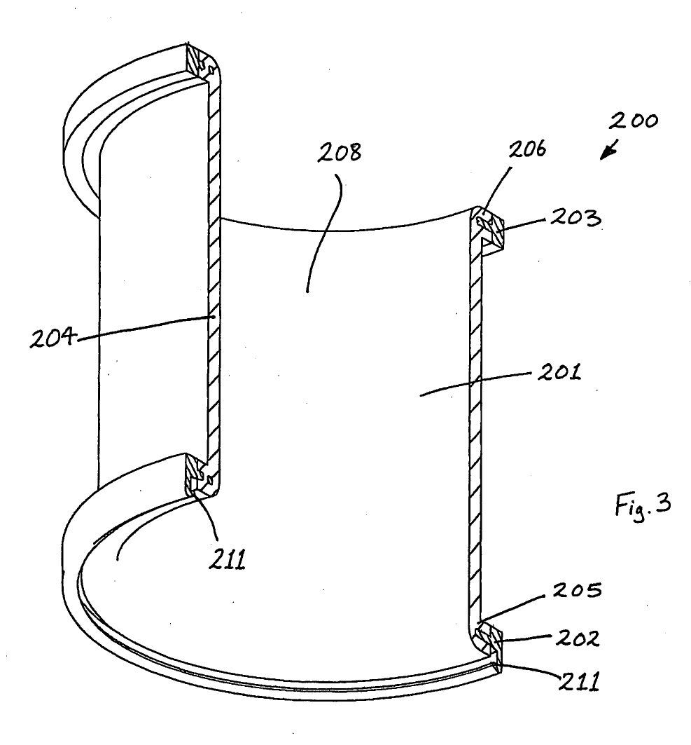

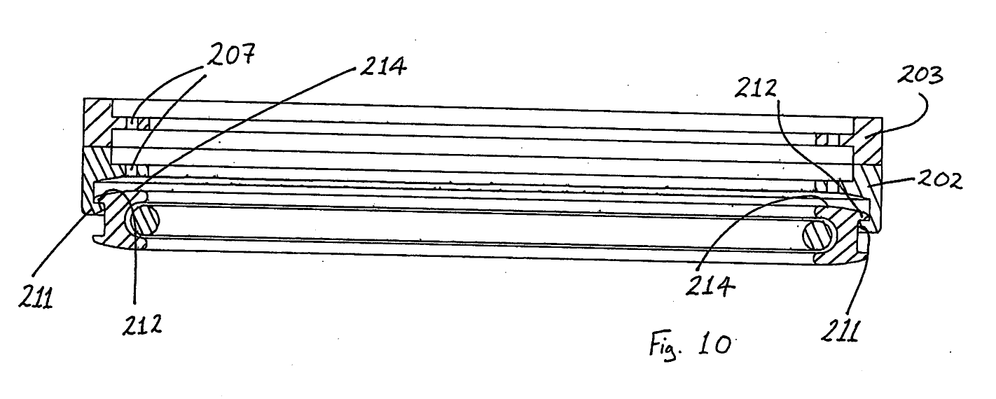

Referring to the drawings, and initially to FIGS. 1 to 11 thereof, there is illustrated a surgical sealing device 200 according to the invention. The device 200 comprises a sealing valve 201 (FIG. 1) for sealing across an opening to an internal cavity, a relatively rigid distal ring element 202, and a relatively rigid proximal ring element 203 (FIG. 2).

The sealing valve 201 comprises a generally cylindrical sealing body 204 having a distal flange 205 at a distal end of the body 204 turned radially outwardly from the cylindrical body 204, and a proximal flange 206 at a proximal end of the body 204 turned radially outwardly from the cylindrical body 204. The sealing valve 201 has a lumen 208 extending therethrough.

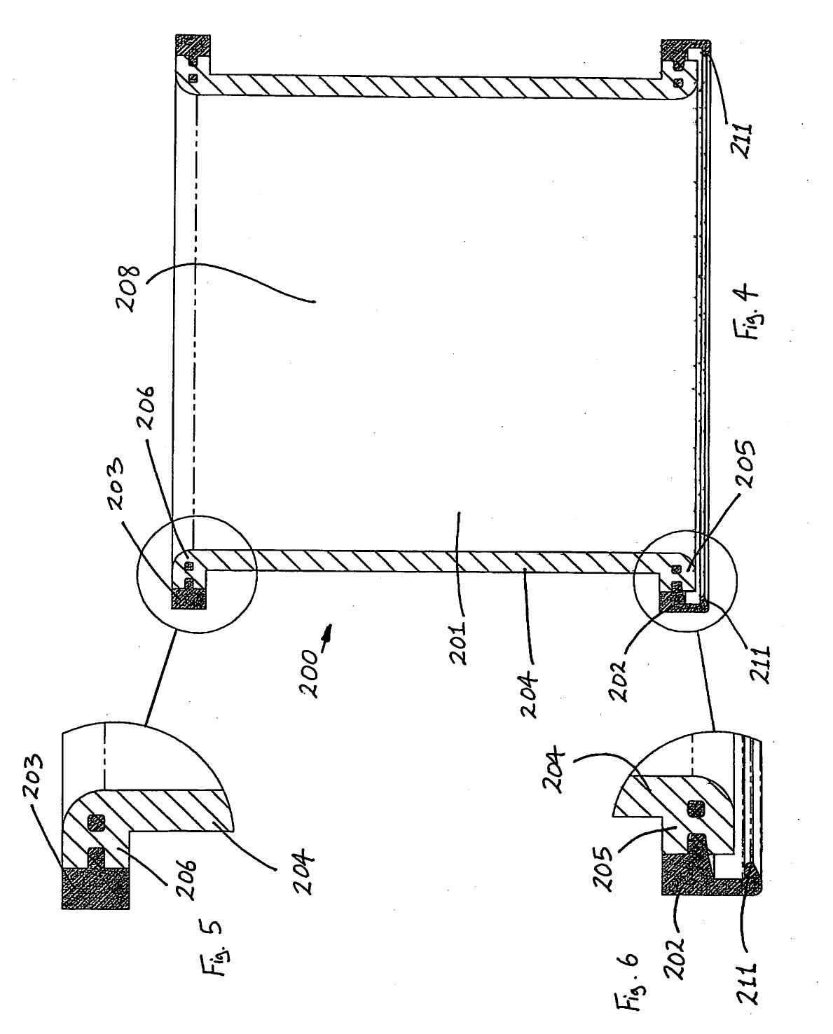

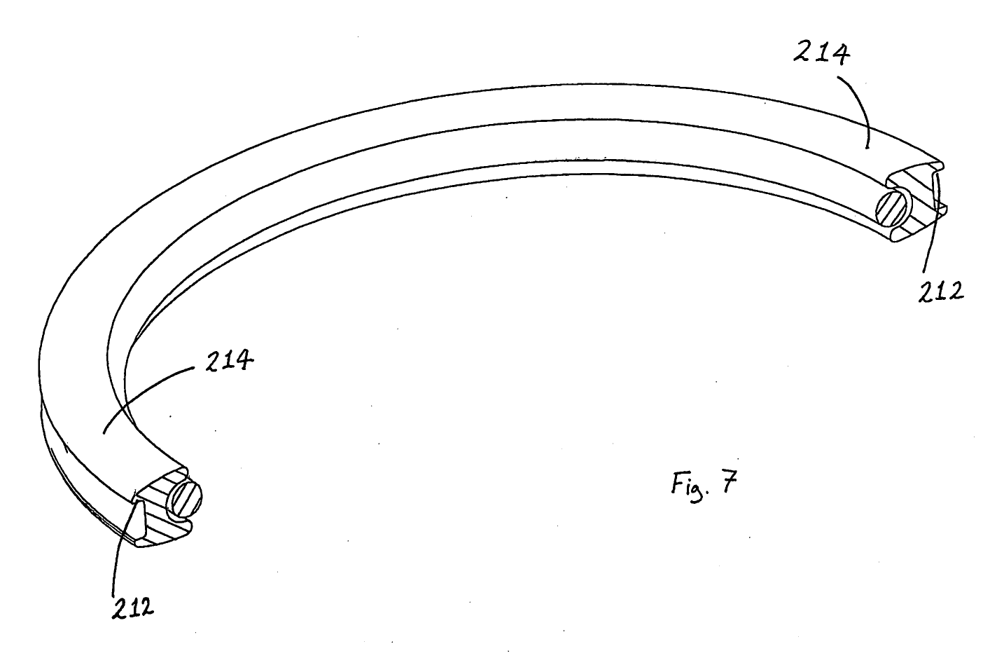

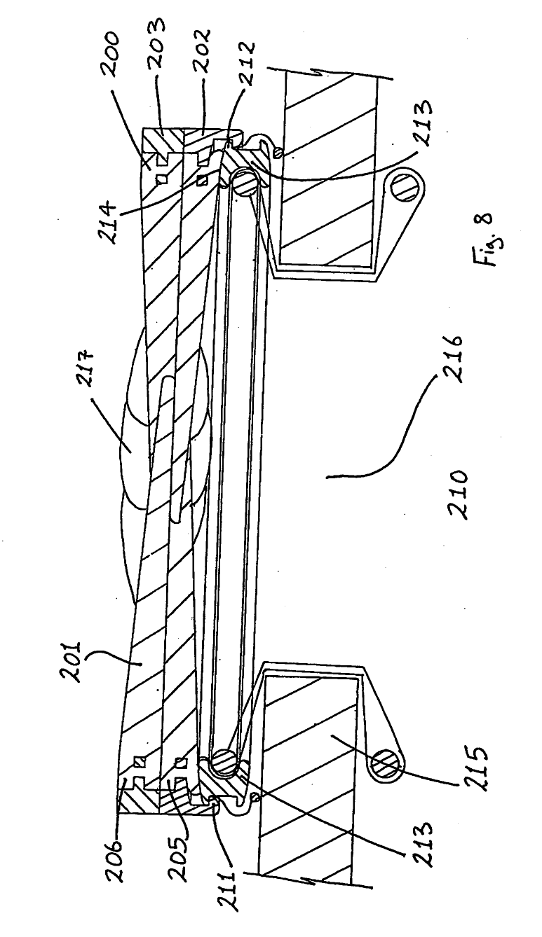

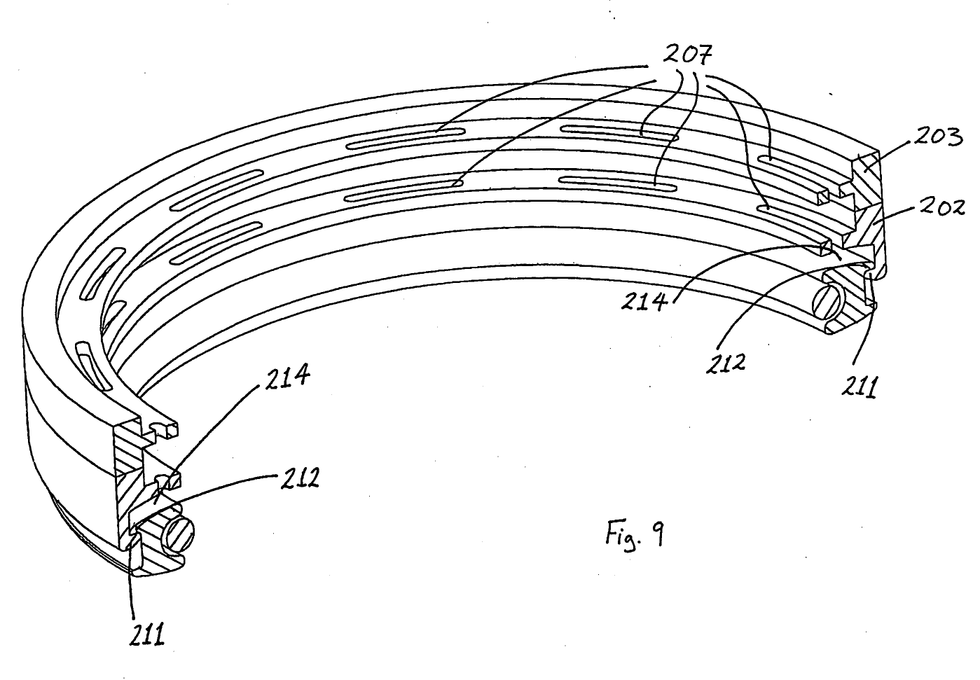

The distal flange 205 is twistable relative to the proximal flange 206 to twist the valve body 204 between an untwisted configuration (FIG. 4) and a twisted configuration (FIG. 8). In the untwisted configuration the lumen 208 is open, and in the twisted configuration, the lumen 208 is closed to seal across an opening to an internal cavity.

The distal ring element 202 is fixedly attached to the distal flange 205 and the proximal ring element 203 is fixedly attached to the proximal flange 206. In this case, overmoulding a part of the valve 201 over a part of the ring elements 202, 203 is used to attach the ring elements 202, 203 to the valve 201. Both of the ring elements 202, 203 have a series of overmould slot openings 207 therein. The openings 207 provide pathways for the material of the valve 201 to mould into and around during the over moulding process. In this manner, a particularly secure attachment of the ring elements 202, 203 to the valve 201 is achieved.

The rigid distal ring element 202 and the rigid proximal ring element 203 provide a convenient means by which the valve 201 may be securely gripped and twisted from the untwisted configuration to the twisted configuration.

However it will be appreciated that the sealing body 204 may alternatively be gripped directly, for example at the flanges 205, 206. It is not necessary to use the ring elements 202, 203 for gripping.

The distal ring element 202 may be coupled to the proximal ring element 203 to maintain the valve 201 in the twisted configuration. For example, the distal ring element 202 may be snap-fitted to the proximal ring element 203 to maintain the distal flange 205 twisted relative to the proximal flange 206. In this manner, the ring elements 202, 203 act as a lock for maintaining the valve 201 in the twisted configuration.

In the twisted configuration (FIG. 8), the distal ring element 202 engages the proximal ring element 201 in an abutting manner, and the distal flange 205 engages the proximal flange 206 in an abutting manner. In this way the valve 201 is constrained longitudinally while in the twisted configuration. In particular, the location of the distal flange 205 is longitudinally fixed relative to the location of the proximal flange 206, while in the twisted configuration.

The valve 201 may be a single piece moulded gel component formed of a gel material. The gel material may include an elastomer, such as silicone or latex. The gel material may also include an oil, and/or a foam.

In one embodiment the valve 201 is of a gelatinous elastomeric material. An extensive review of gelatinous elastomeric materials is included in U.S. Pat. No. 5,994,450 (Pearce), the entire contents of which are incorporated herein by reference. One such group of gelatinous elastomers may comprise a triblock copolymer A-B-A wherein

A is selected from the group consisting of monoalkenylarene polymers and B is a hydrogenated polymer including a plurality of isoprene monomers and a plurality of butadiene monomers. The material includes a plasticiser which may be selected from the group consisting of naturally derived oils, synthetic oils and liquid oligomers. The materials required to form a suitable gel material are available, for example, from the company Crinnis of USA.

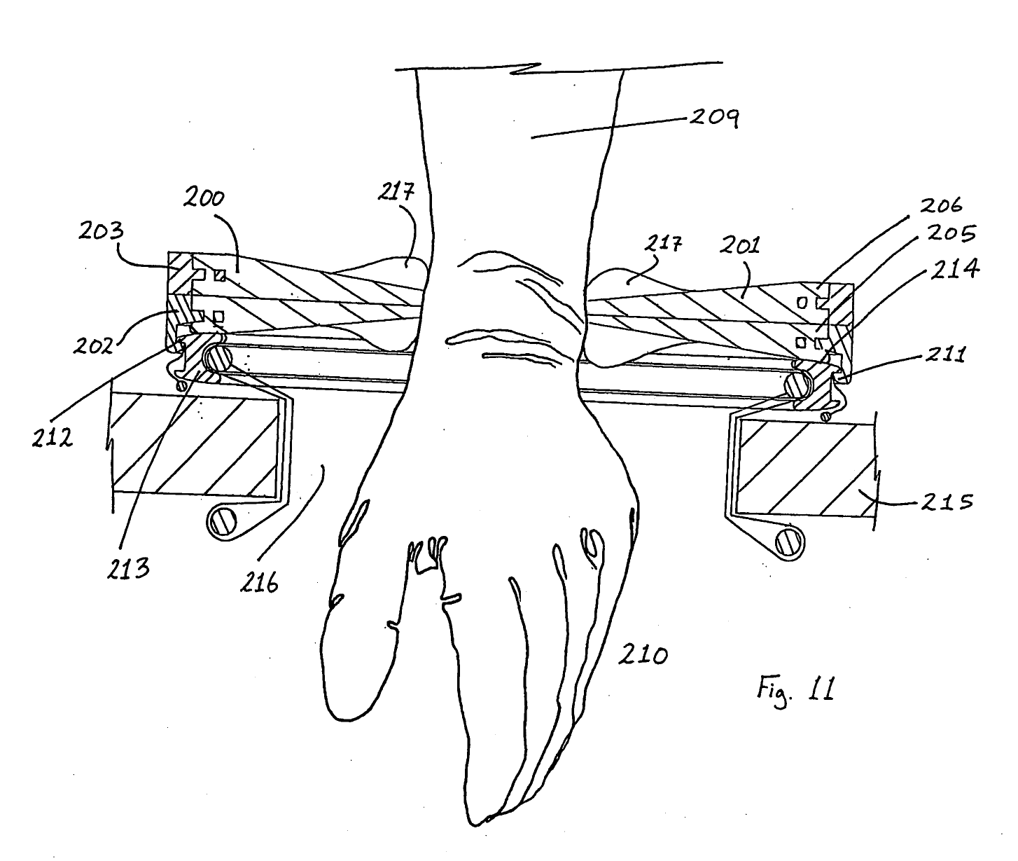

The highly flexible nature of the gelatinous elastomeric material enables the opposing sidewalls of the lumen 208 to be forced radially part to partially open the lumen 208, while the valve 201 remains in the twisted configuration and the distal flange 205 remains twisted relative to the proximal flange 206. By partially opening the lumen 208 this enables an object, such as a surgeon's hand and/or forearm 209, or a surgical instrument, to pass through the lumen 208 to access an internal cavity 201 (FIG. 11).

With the surgical sealing device 200 of the invention, it is not necessary to untwist the valve 201 from the twisted configuration to the untwisted configuration to open the lumen 208. The lumen 208 may be partially opened by forcing the sidewalls of the valve 201 radially apart while the valve 201 remains twisted. Because the valve 201 remains twisted, the loss of insufflation gases from the abdomen during insertion of an object through the lumen 208 is minimised.

One means of forcing the sidewalls of the valve 201 apart to open the lumen 208 is to engage the object, such as the surgeon's hand and/or forearm 209 or the surgical instrument, with the closed lumen 208, and pass the object through the lumen 208 to force the sidewalls radially apart.

Because the valve 201 is longitudinally constrained in the twisted configuration, no longitudinal movement of the valve 201 occurs while in the twisted configuration. In particular opening of the lumen 208, while the valve 201 remains in the twisted configuration, does not involve longitudinal movement of the flanges 205, 206 or of the ring elements 202, 203. Rather, in the twisted configuration, the lumen 208 is opened by forcing the opposing sidewalls radially apart. This arrangement results in an extremely compact and low profile surgical sealing device.

The gelatinous elastomeric material is also highly resilient. This causes the lumen 208 to be biased towards being closed while in the twisted configuration. Thus the valve 201 effects a seal between the sidewalls of the lumen 208 and the object inserted through the lumen 208 (FIG. 11). Upon withdrawal of the object from the lumen 208, the resilient nature of the valve 201 closes the lumen 208 to prevent loss of insufflation gases from the abdomen.

The distal ring element 202 has a radially inwardly protruding lip formation 211. The lip formation 211 may be engaged under a corresponding shoulder formation 212 of a wound retractor device 213 to facilitate snap-fit mounting of the surgical sealing device 200 to the wound retractor device 213 (FIG. 8).

The distal flange 205 protrudes distally to engage against a proximal surface 214 of the wound retractor device 213 when the surgical sealing device 200 is mounted thereto, as illustrated in FIG. 11. In this manner, the surgical sealing device 200 effects a seal between the surgical sealing device 200 and the wound retractor device 213, upon mounting.

The distal ring element 202 is suitable for mounting the surgical sealing device 200 to a variety of wound retractor devices, such as the wound retractor devices described in U.S. Pat. No. 6,582,364 and/or the U.S. patent application published under No. 2001/0037053 and/or U.S. patent application Ser. No. 10/678,653, the relevant contents of which are incorporated herein by reference.

The device 200 may be formed by locating the distal ring element 202 at one end of a generally cylindrical mould and locating the proximal ring element 203 at an opposite end of the mould. The gelatinous elastomeric material in a liquid state is then passed into the mould and allowed to set. In this manner the distal flange 205 is overmoulded over the distal ring element 202 and the proximal flange 206 is overmoulded over the proximal ring element 203. In addition the body 204 of the valve 201 is formed into the cylindrical shape.

After the gelatinous elastomeric material has set, the device 200 may be removed from the mould. By gripping and twisting the ring elements 202, 203, the valve 201 may be twisted from the untwisted configuration. The ring elements 202, 203 may then be snap-fitted together to maintain the valve 201 in the twisted configuration.

In use, an incision is made in the body tissue 215, and the wound retractor device 213 is employed to retract the sides of the incision to reveal an opening 216 to the internal cavity 210. The surgical sealing device 200 with the valve 201 pre-twisted into the twisted configuration is then mounted to the wound retractor device 213 by snap-fitting the lip formation 211 beneath the shoulder formation 212 (FIG. 8). In this case, the surgical sealing device 200 is located externally of the internal cavity 210 proximally of the opening 216, and adjacent to the proximal end of the wound retractor device 213.

The lumen 208 is closed in the twisted configuration to prevent loss of insufflation gases from the internal cavity 210. The pleats 217 formed upon twisting of the valve 201 assist in maintaining a gas-tight seal.

To access the internal cavity 210 through the lumen 208, the object, such as the surgeon's hand/forearm 209 or the surgical instrument, is engaged with the closed lumen 208 and forced distally through the lumen 208. Due to the highly elastic nature of the gelatinous elastomeric material, the sidewalls of the lumen 208 are forced radially apart to partially open the lumen 208. The object may therefore pass through the lumen 208 and access the internal cavity 210 (FIG. 11).

The sidewalls of the lumen 208 effect a seal to the object inserted through the lumen 208 to prevent loss of insufflation gases from the abdomen. The pleats 217 assist in maintaining the gas-tight seal.

When the object is withdrawn from the lumen 208, the resilient nature of the valve 201 causes the lumen 208 to close once more.

It is not necessary to untwist the valve 201, or to move the flanges 205, 206 or the ring elements 202, 203 longitudinally to open the lumen 208. The surgical sealing device 200 therefore minimises the loss of insufflation gases.

The valve 201 is preferably pre-twisted into the twisted configuration during manufacture of the valve 201, and is supplied for surgical use in the twisted configuration with the ring elements 202, 203 snap-fitted together to maintain the valve 201 in the twisted configuration. In this case, the surgeon is not required to twist the valve 201 before use, and may therefore quickly and simply use the pre-twisted surgical sealing device 200 in the desired surgical procedure.

Alternatively the valve 201 may be supplied in the untwisted configuration. In this case, the surgeon twists the valve 201 into the twisted configuration, and snap-fits the ring elements 202, 203 together to maintain the valve 201 in the twisted configuration before use. By supplying the valve 201 in the untwisted configuration this may minimise the possibility of material degradation during storage of the surgical sealing device 1.

It will be appreciated that the ring elements 202, 203 may be fixedly attached to the flanges 205, 206 in a variety of possible alternative manners. For example the ring elements 202, 203 may be fixedly attached to the flanges 205, 206 by means of an adhesive, or by means of a mechanical keying arrangement.

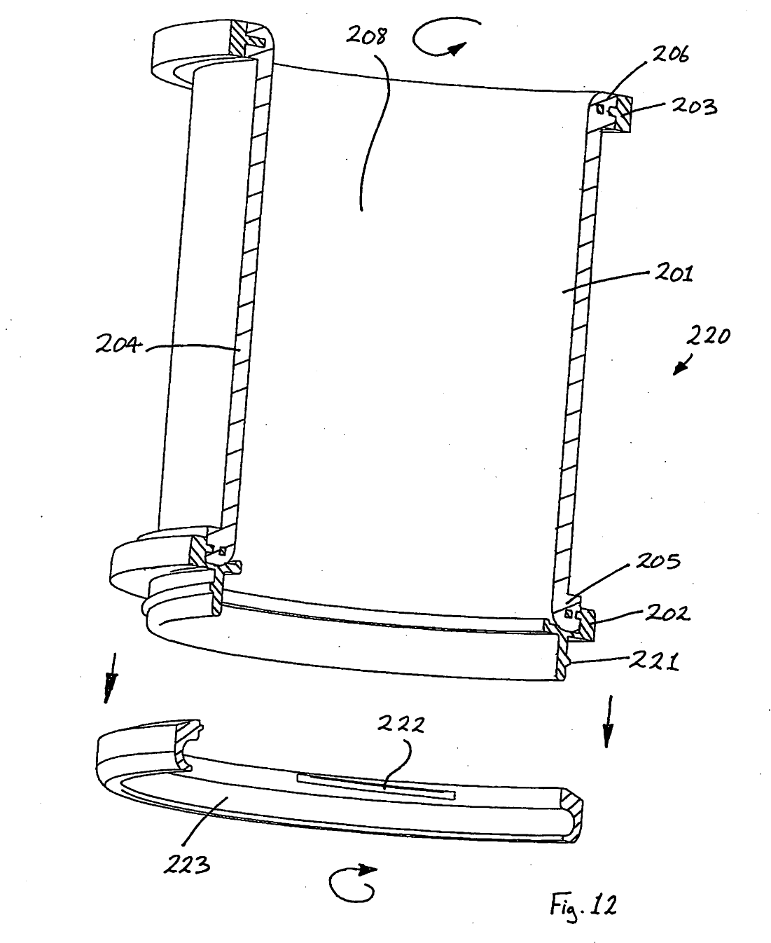

It will also be appreciated that the surgical sealing device may be mounted to the wound retractor device 213 in a variety of possible alternative manners. For example, FIG. 12 illustrates another surgical sealing device 220 according to the invention, which is similar to the device 200 of FIGS. 1 to 11, and similar elements in FIG. 12 are assigned the same reference numerals. In this case the distal ring element 202 has a screw-thread mounting of the surgical sealing device 220 to a proximal ring element 223 of the wound retractor device. The proximal ring element 223 has a corresponding screw-thread formation 222.

In certain applications the valve 201 may alternatively be formed as a single piece moulded rubber or elastomeric component.

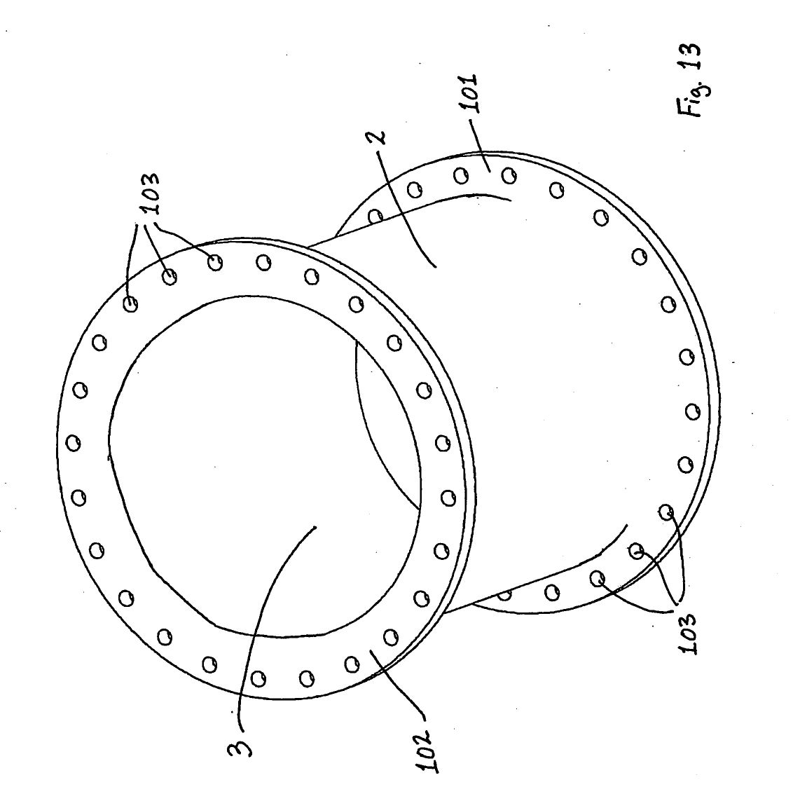

Referring to FIGS. 13 to 17, there is illustrated a further surgical sealing device 100 according to the invention, which is similar to the device 200 described previously with reference to FIGS. 1 to 11.

In this case the sealing valve comprises the generally cylindrical sealing body part 2, a distal end region 101 and a proximal end region 102. As illustrated in FIG. 13, the distal end region 101 and the proximal end region 102 are provided in the form of two flanges turned radially outwardly relative to the generally cylindrical sealing body 2. In this case the distal end region 101 and the proximal end region 102 are turned outwardly substantially perpendicular to the generally cylindrical sealing body 2. A plurality of female recesses 103 are provided spaced around the distal end region 101 and the around the proximal end region 102.

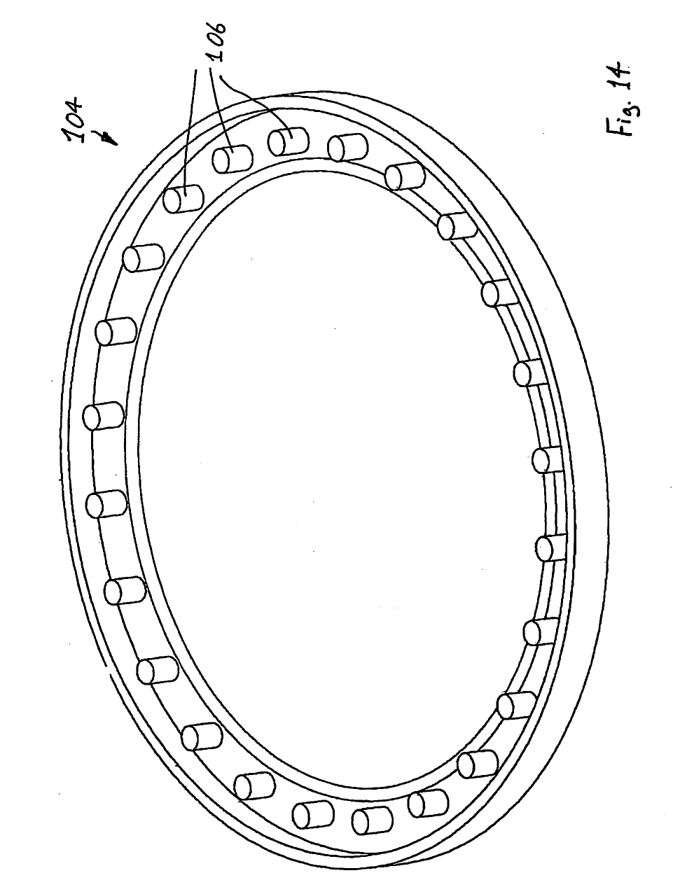

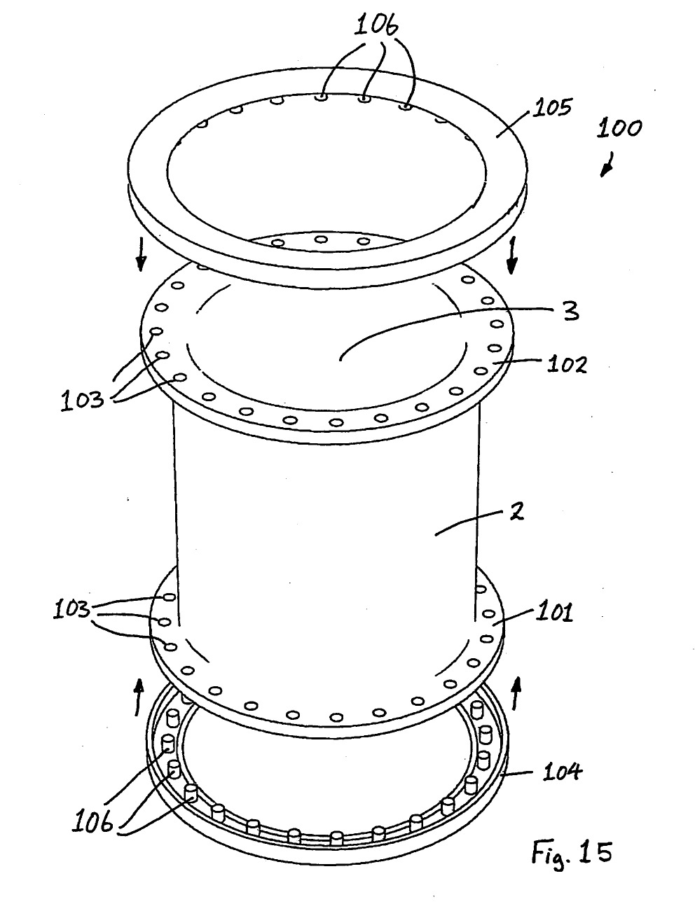

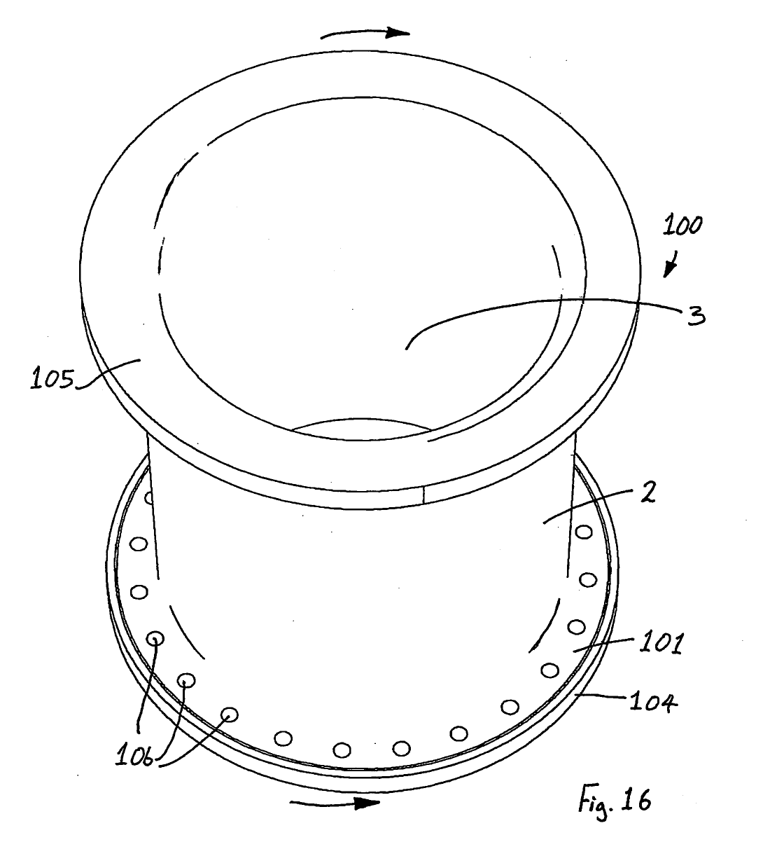

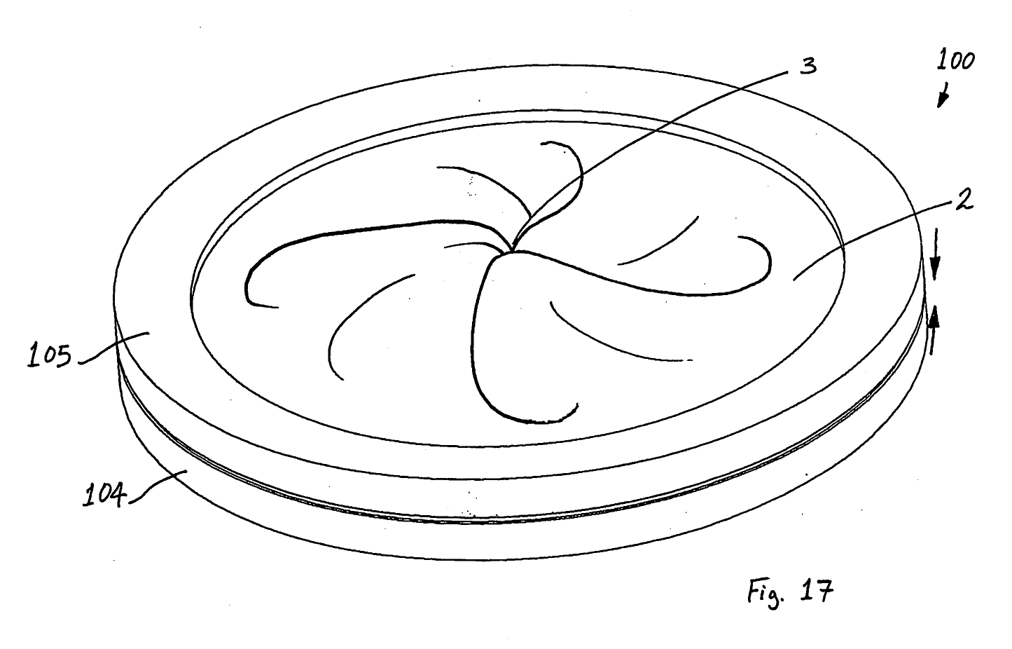

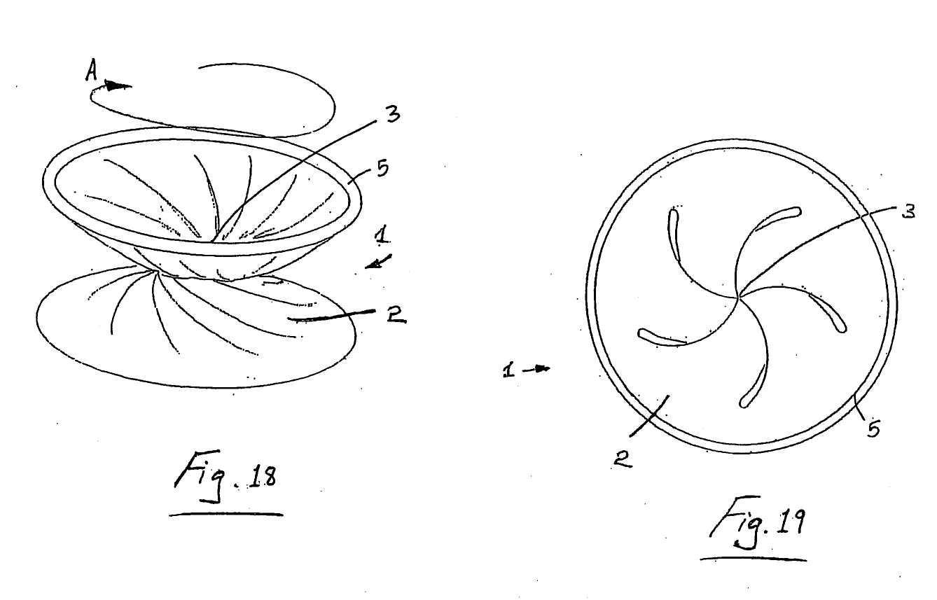

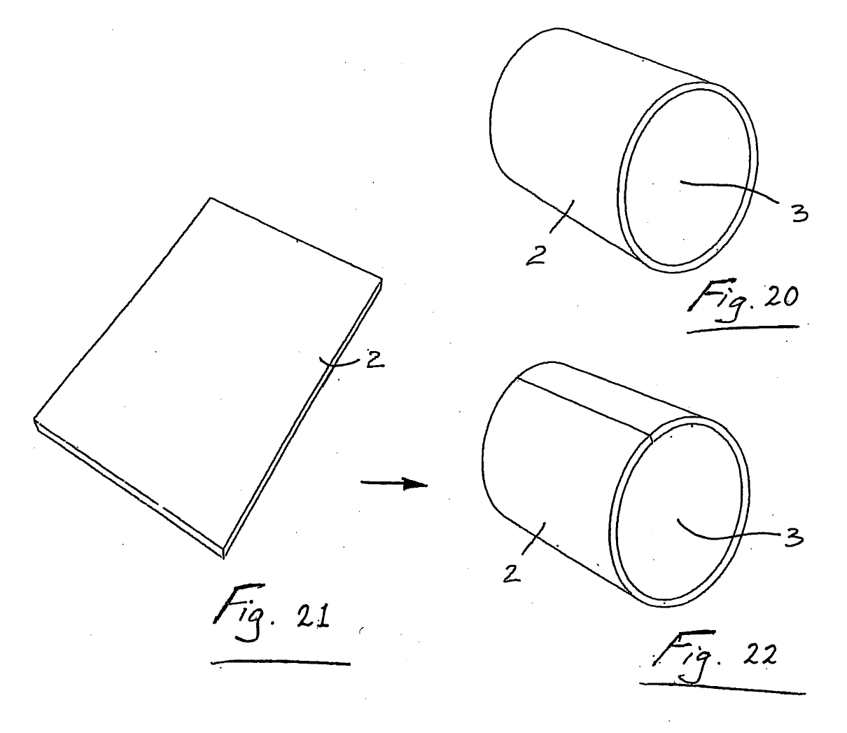

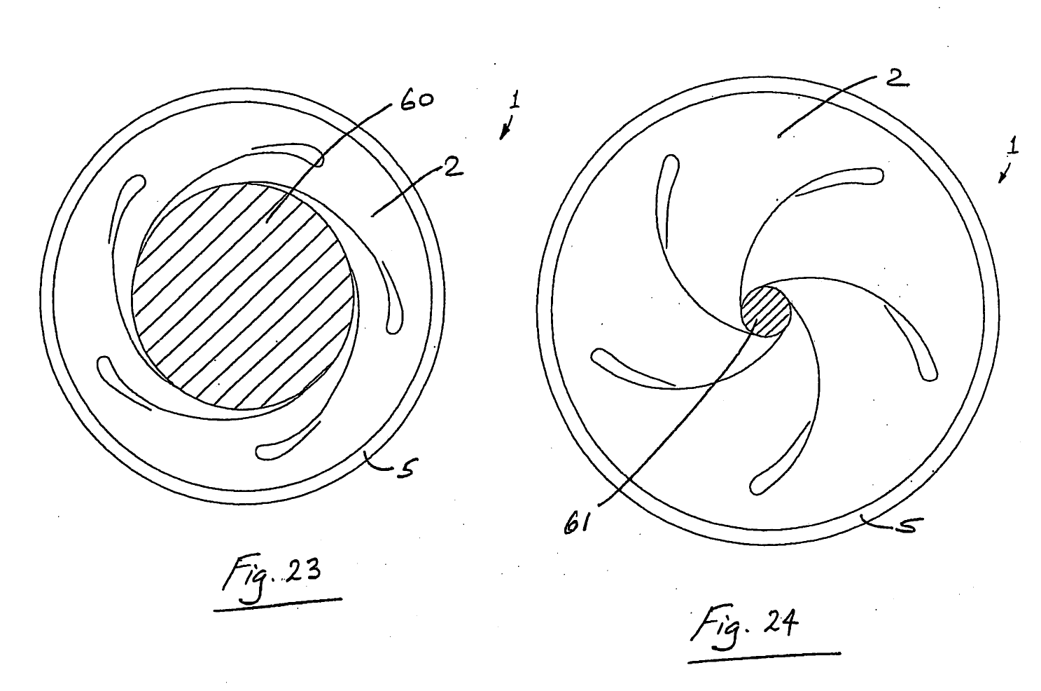

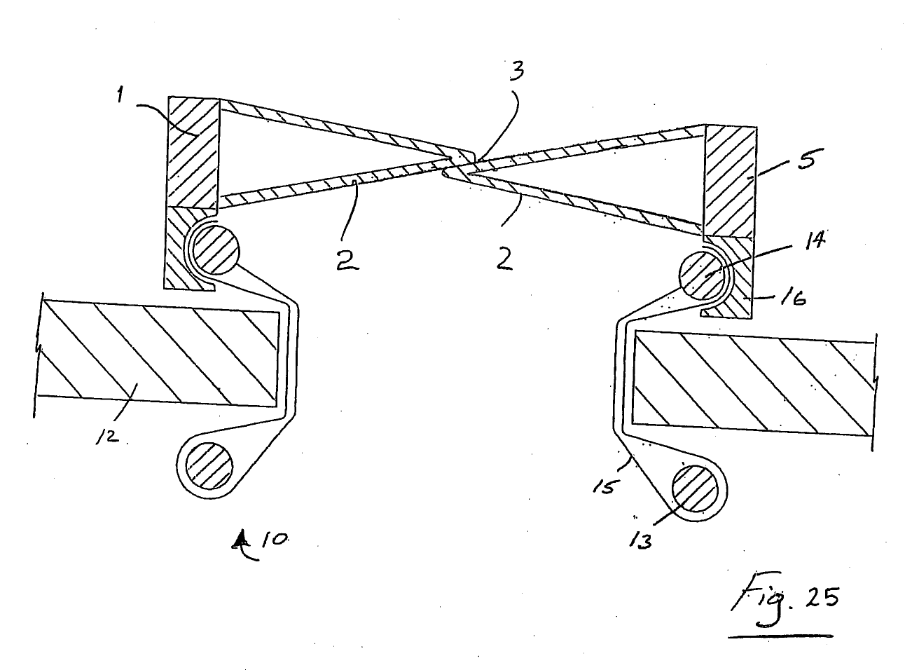

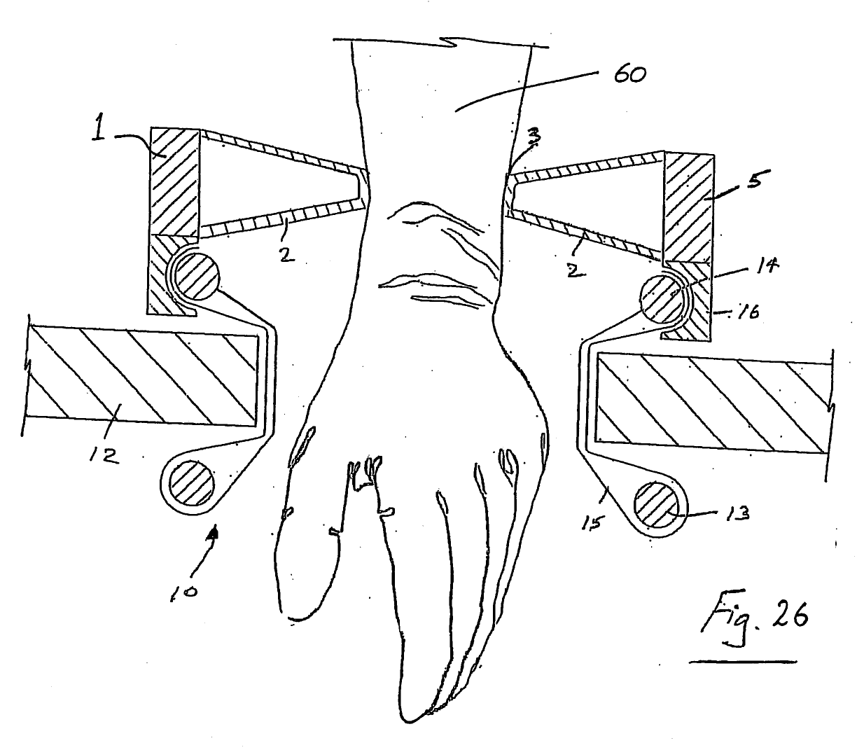

The device 100 also comprises a distal end element 104 and a proximal end element 105 (FIGS. 14 and 15). Each of the distal end element 104 and the proximal end element 105 are provided in the form of a ring element having a plurality of protruding male elements 106 upstanding from the ring element (FIG. 14). By co-operatively engaging the male elements 106 in the female recesses 103, the distal end element 104 may be. fixedly attached to the distal end region 101 of the sealing valve, and the proximal end element 105 may be fixedly attached to the proximal end region 102 of the sealing valve, as illustrated in FIGS. 15 and 16.

The end elements 104, 105 are rigid relative the elastomeric sealing valve. Thus, by attaching the rigid end elements 104, 102 to the end regions 101, 102 of the sealing valve, the end regions 101, 102 of the sealing valve are reinforced. The reinforced end regions 101, 102 are therefore easier for a user to grip and rotate so that the generally cylindrical sealing body 2 may be twisted from the untwisted configuration (FIG. 16) to the twisted configuration (FIG. 17).

When the generally cylindrical sealing body 2 is in the twisted configuration, the distal end element 104 and the proximal end element 105 may be snap-fit together (FIG. 17). By snap-fitting the end elements 104, 105 together, the position of the distal end region 101 of the sealing valve will be maintained relative to the position of the proximal end region 102 of the sealing valve. In this way, the end elements 104, 105 acts as a clock to maintain the generally cylindrical sealing body 2 will be maintained in the twisted configuration.

It will be appreciated that the distal end elements 104 may be employed as a mounting element for mounting the device 100 to a wound retractor device, in a manner similar to that described previously with reference to FIGS. 1 to 11.

Referring to FIGS. 18 to 26, there is illustrated another surgical sealing device 1 according to the invention, which is similar to the device 200 described previously with reference to the FIGS. 1 to 11. The device 1 is suitable for use in a surgical procedure, such as hand assisted laparoscopic surgery. The device I is a twist valve formed by a sealing body of elastomeric material. The sealing body has an accessway 3 for passage of an object, such as a surgeon's hand/forearm or a surgical instrument, therethrough. The accessway 3 is defined by the twist of the sealing body. The accessway 3 has a normally closed configuration as illustrated in FIGS. 18 and 19 in which the walls of the sealing body such as a surgeon's hand/arm or instrument are interengaged. The accessway 3 is opened up on passage of an object therethrough while maintaining sealing engagement between the object and the sealing body.

The sealing body may be of any suitable elastomeric material. It may comprise silicone, latex, rubber, neoprene or the like and may be in the form of a gel.

Referring to FIG. 20 the valve 1 is formed from a generally cylindrical body 2 of elastomeric, in this case gel, material. The cylindrical body 2 is in this case formed by moulding or extrusion. The twist valve 1 is formed by twisting the cylindrical body 2 as indicated by the arrow in FIG. 18. To maintain the body 2 in the twisted configuration, it is retained by a mounting frame 5. The mounting frame 5 also provides a coupling for releasable or permanent coupling to another device such as a retractor.

Alternatively, as illustrated in FIGS. 21 and 22, the cylindrical sealing body 2 may be formed by folding a generally flat sheet of material into the form of a cylinder.

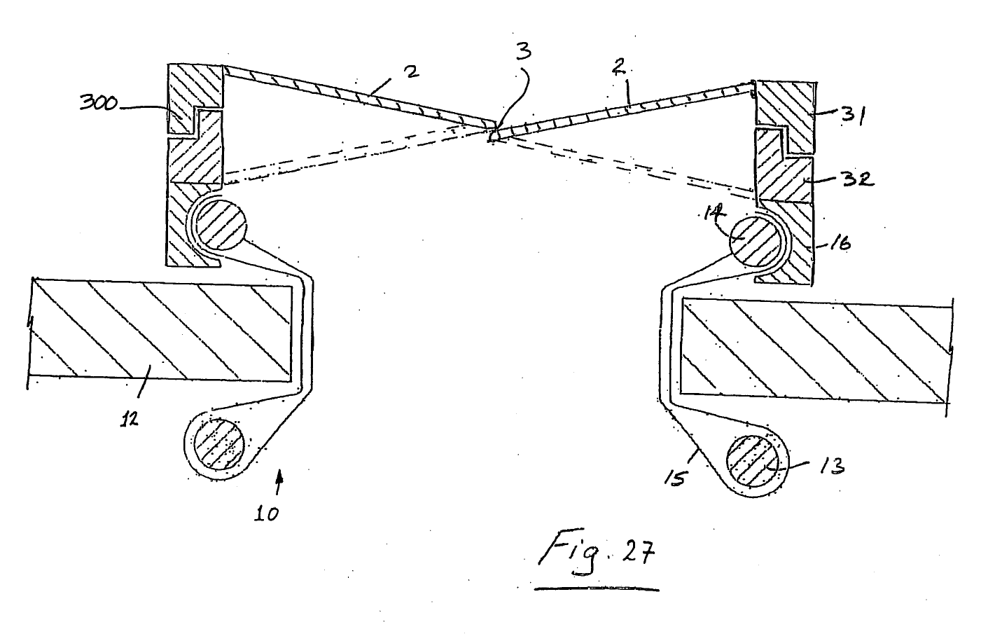

FIGS. 23 and 24 illustrate the gel twist valve 1 according to the invention in use. In the case of a relatively large object, such as surgeon's hand/arm 60, the gel twist valve 1 can readily stretch to accommodate the object while still maintaining excellent sealing engagement between the valve 1 and the object 60. In the case of smaller diameter objects, such as a surgical instrument 61, excellent sealing engagement is maintained even if the object is shifted significantly transversely of the valve 1 during use.

Referring to FIGS. 25 and 26 there is illustrated the device of the type described above, which in this case is coupled to a wound retractor device 10 located in an incision at a surgical site such as in an abdominal wall 12. The retractor 10 may be of any suitable construction such as the retractors described in our U.S. 2001/0037053A and/or U.S. Pat. No. 6,582,364, the whole contents of which are incorporated herein by reference. The valve 1 may be coupled to the retractor 10 by any suitable means, such as by means of a screw-thread arrangement, or by means of a snap-fit arrangement.

In this case the retractor 10 has a distal ring 13 and a proximal ring 14 with a retracting sleeve 15 extending therebetween, as illustrated in FIG. 25. The retractor 10 also has a proximal guide or mounting ring 16. In this case the proximal ring 14 is located in a recess in the guide/mounting ring 16 however various other constructions are possible. In FIGS. 25 and 26, the valve 1 is illustrated mounted adjacent to a proximal end of the retractor 10, in this case to the mounting ring 16. However, in some cases the valve I may be mounted adjacent to a distal end of the retractor 10. The valve 1 may also be mounted to the retractor 10 with the valve 1 located within the wound opening.

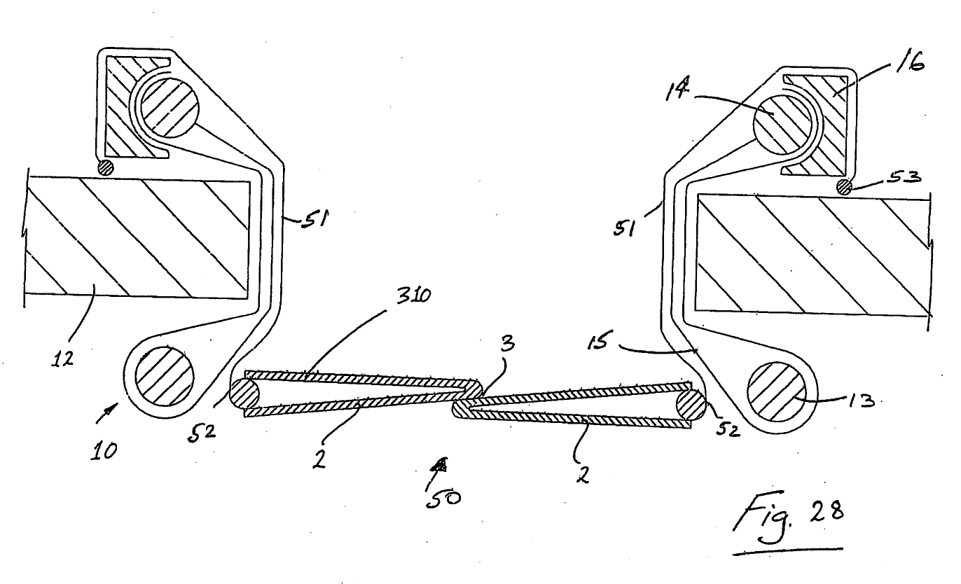

Referring to FIG. 27 there is illustrated another surgical sealing device 300 according to the invention, which is similar to the device 1 of FIGS. 18 to 26, and similar elements In FIG. 27 are assigned the same reference numerals.

In this case there are upper and lower mountings 31, 32 which may be movable relative to one another to facilitate formation of the twist. The mountings 31, 32 may be movable in use to adjust the twist, for example to accommodate different sized objects.

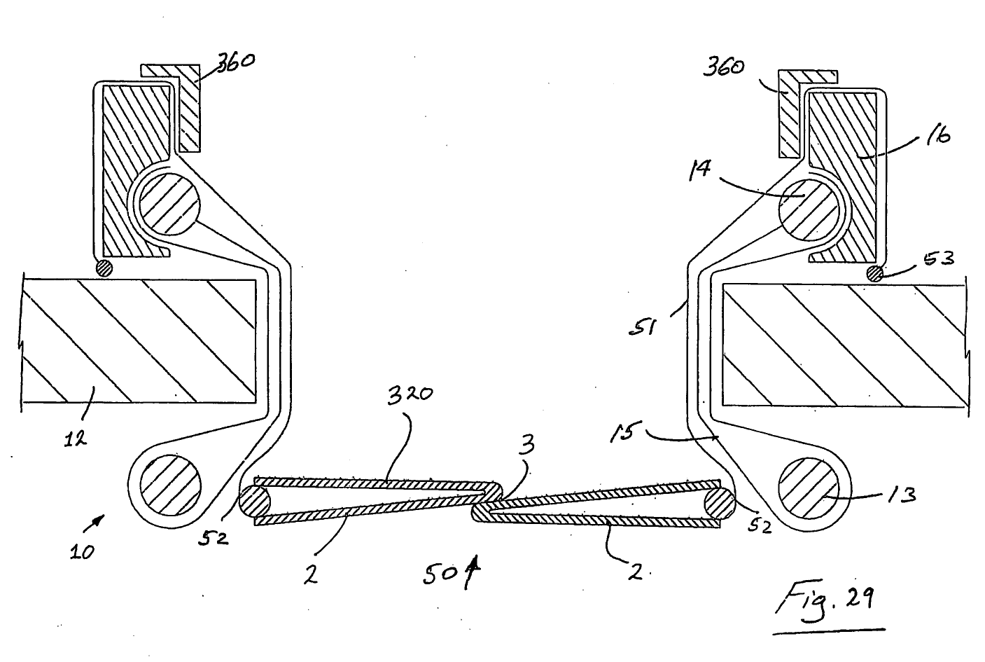

Referring to FIG. 28 there is illustrated a further surgical sealing device 310 according to the invention which is similar to the device 1 of FIGS. 18 to 26, and similar elements In FIG. 28 are assigned the same reference numerals.

In this case, in use, a gel twist valve 50 is mounted internally in the abdomen distally of the wound opening adjacent to the distal end of the retractor 10 to provide an extremely low profile device 310 and to provide the accessway 3 even closer to the location of the surgical procedure. A flexible attachment sleeve 51 is attached at a distal end 52 to the valve 50 and extends externally of the incision in the abdomen for attachment to a fixture, such as an outer proximal ring 16 of the retractor 10. In this case the sleeve 51 has a proximal mounting O-ring 53 at a proximal end which is attached to the retractor ring 16, for example by folding the sleeve 51 over the ring 16 and hooking the sleeve O-ring 53 underneath the retractor ring 16.

Referring now to FIG. 29 there is illustrated another surgical sealing device 320 according to the invention which is similar to the device 310 of FIG. 8 and like parts in FIG. 29 are assigned the same reference numerals.

In this case an additional locking or sealing member such as a sealing ring 360 is used to press the attachment sleeve 51 into sealing engagement with the proximal retractor ring 16. This arrangement further enhances the sealing of the attachment sleeve 51 to substantially prevent any leakage of pneumoperitoneum gas.

The surgical sealing device of the invention provides sealed access without risk of gas leakage from the abdominal cavity.

The gel material of the twist valve has a high level of elasticity. It also has a relatively small thickness and volume. On twisting to form the twist valve, the creases formed in the elastomeric body assist in providing enhanced support. The twist valve is effectively completely closed in the rest position thus providing no leak path for insufflation gases from the abdomen. The twist valve can be readily opened by a user inserting his fingers and then twisting them against the twist to open the accessway in a substantially leak-tight manner. Alternatively the twist valve may be pushed down to initially open the accessway. In this way the material of the twist valve moulds to the object inserted therethrough to provide a seal substantially preventing leakage.

The invention is not limited to the embodiments hereinbefore described, with reference to the accompanying drawings, which may be varied in construction and detail.

1. A surgical sealing device comprising a sealing valve for sealing across an opening to an internal cavity, the sealing valve having a material which is twisted to form a seal, the seal being maintained upon passage of an object therethrough.

2. A device as claimed in claim 1 wherein the material defines a lumen extending therethrough, the lumen being closed when the material is twisted to form the seal.

3. A device as claimed in claim 2 wherein the material is configured to enable the lumen to be at least partially opened for passage of an object through the lumen to access the internal cavity, while the lumen remains twisted.

4. A surgical sealing device comprising a sealing valve for sealing across an opening to an internal cavity;
the sealing valve having a material defining a lumen;
the material being twisted to close the lumen;
the lumen being at least partially opened upon passage of an object through the lumen.

5. A device as claimed in claim 4 wherein the material sealingly engages the object upon passage therethrough.

6. A device as claimed in claim 4 wherein the lumen is at least partially opened while the material remains twisted.

7. A device as claimed in claim 1 wherein a first part of the material is twistable relative to a second part of the material between an untwisted configuration and a twisted configuration.

8. A device as claimed in claim 7 wherein in the untwisted configuration, the lumen is open.

9. A device as claimed in claim 7 wherein in the twisted configuration, the lumen is closed.

10. A device as claimed in claim 1 wherein the valve is at least partially of a gelatinous elastomeric material.

11. A device as claimed in claim 7 wherein the first part of the valve comprises a distal end part of the valve.

12. A device as claimed in claim 7 wherein the second part of the valve comprises a proximal end part of the valve.

13. A device as claimed in claim 7 wherein, in the twisted configuration, a third part of the valve is movable relative to a fourth part of the valve to at least partially open the lumen.

14. A device as claimed in claim 13 wherein the third part of the valve is movable substantially radially relative to the fourth part of the valve.

15. A device as claimed in claim 13 wherein the third part of the valve comprises a part of a sidewall of the lumen.

16. A device as claimed in claim 15 wherein the fourth part of the valve comprises an opposing part of the sidewall of the lumen.

17. A device as claimed in claim 7 wherein, in the twisted configuration, the valve is constrained longitudinally.

18. A device as claimed in claim 17 wherein, in the twisted configuration, the location of the first part of the valve is longitudinally fixed relative to the location of the second part of the valve.

19. A device as claimed in claim 18 wherein, in the twisted configuration, the first part of the valve engages the second part of the valve.

20. A device as claimed in claim 7 wherein, in the twisted configuration, the lumen is at least partially openable upon engagement of an object with the closed lumen and passage of the object through the lumen.

21. A device as claimed in claim 7 wherein, in the twisted configuration, the valve is configured to effect a seal between a sidewall of the lumen and an object inserted through the lumen.

22. A device as claimed in claim 7 wherein, in the twisted configuration, the lumen is biased towards being closed.

23. A device as claimed in claim 22 wherein the valve is of a resilient material to bias the lumen towards being closed.

24. A device as claimed in claim 7 wherein the valve comprises a generally cylindrical sealing body which is twistable into the twisted configuration.

25. A device as claimed in claim 24 wherein the generally cylindrical sealing body is formed by moulding or extrusion.

26. A device as claimed in claim 24 wherein the generally cylindrical sealing body is formed from a generally flat sheet of material.

27. A device as claimed in claim 7 wherein the first part of the material and/or the second part of the material is turned radially outwardly.

28. A device as claimed in claim 27 wherein the first part of the material and/or the second part of the material is provided in the form of a flange.

29. A device as claimed in claim 7 wherein the device comprises a lock for maintaining the valve in the twisted configuration.

30. A device as claimed in claim 29 wherein the lock comprises a first lock part attached to the first part of the valve and a second lock part attached to the second part of the valve, the first lock part being coupleable to the second lock part to maintain the first part of the valve twisted relative to the second part of the valve.

31. A device as claimed in claim 30 wherein the first lock part and the second lock part are configured to be snap-fitted together.

32. A device as claimed in claim 30 wherein the lock part is attached to the part of the valve by overmoulding a part of the valve over at least part of the lock part.

33. A device as claimed in claim 32 wherein the lock part comprises one or more overmould openings therein.

34. A device as claimed in claim 30 wherein the lock part is attached to the part of the valve by a mechanical attachment arrangement.

35. A device as claimed in claim 34 wherein the mechanical attachment arrangement comprises one or more protruding male elements for co-operative reception in one or more female recesses.

36. A device as claimed in claim 35 wherein the lock part comprises the one or more male elements and the part of the valve comprises the one or more female recesses.

37. A device as claimed in claim 30 wherein the lock part is rigid relative to the valve.

38. A device as claimed in claim 30 wherein the lock part comprises a ring element.

39. A device as claimed in claim 1 wherein the device comprises a grip element for gripping of the valve.

40. A device as claimed in claim 39 wherein the device comprises a first grip element attached to the first part of the valve and a second grip element attached to the second part of the valve.

41. A device as claimed in claim 40 wherein the grip element is attached to the part of the valve by overmoulding a part of the valve over at least part of the grip element.

42. A device as claimed in claim 41 wherein the grip element comprises one or more overmould openings therein.

43. A device as claimed in claim 40 wherein the grip element is attached to the part of the valve by a mechanical attachment arrangement.

44. A device as claimed in claim 43 wherein the mechanical attachment arrangement comprises one or more protruding male elements for co-operative reception in one or more female recesses.

45. A device as claimed in claim 44 wherein the grip element comprises the one or more male elements and the part of the valve comprises the one or more female recesses.

46. A device as claimed in claim 39 wherein the grip element is rigid relative to the valve.

47. A device as claimed in claim 39 wherein the grip element comprises a ring element.

48. A device as claimed in claim 1 wherein the device is mountable to a retractor device.

49. A device as claimed in claim 48 wherein the device comprises a mounting element for mounting the device to a retractor device.

50. A device as claimed in claim 49 wherein the mounting element is fixedly attached to the valve.

51. A device as claimed in claim 50 wherein a part of the valve is overmoulded over at least part of the mounting element.

52. A device as claimed in claim 51 wherein the mounting element comprises one or more overmould openings therein.

53. A device as claimed in claim 50 wherein the mounting element is attached to the valve by a mechanical attachment arrangement.

54. A device as claimed in claim 53 wherein the mechanical attachment arrangement comprises one or more protruding male elements for co-operative reception in one or more female recesses.

55. A device as claimed in claim 54 wherein the mounting element comprises the one or more male elements and the valve comprises the one or more female recesses.

56. A device as claimed in claim 49 wherein the mounting element comprises an engagement formation for snap-fit mounting of the device to retractor device.

57. A device as claimed in claim 49 wherein the mounting element comprises a screw-thread formation for screw-thread mounting of the device to a retractor device.

58. A device as claimed in claim 49 wherein the mounting element is rigid relative to the valve.

59. A device as claimed in claim 49 wherein the mounting element comprises a ring element.

60. A device as claimed in claim 49 wherein the device is configured to effect a seal between the device and a retractor device upon mounting of the device to the retractor device.

61. A device as claimed in claim 60 wherein at least part of the valve is configured to engage a retractor device upon mounting.

62. A device as claimed in claim 49 wherein the mounting. element comprises a sleeve for extending between the valve and a retractor device.

63. A device as claimed in claim 62 wherein the sleeve is hookable around a part of a retractor device to mount the device to the retractor device.

64. A device as claimed in claim 63 wherein the sleeve comprises a mounting ring for hooking around a part of a retractor device.

65. A device as claimed in claim 64 wherein the mounting ring is an O-ring.

66. A device as claimed in claim 62 wherein a distal end of the sleeve is coupled to the valve and a proximal and of the sleeve is coupleable to a retractor device.

67. A device as claimed in claim 62 wherein the device comprises a sealing element for sealingly engaging the mounting element to a retractor device.

68. A device as claimed in claim 67 wherein the sealing element comprises a sealing ring.

69. A device as claimed in claim 10 wherein the gelatinous elastomeric material comprises a plasticiser.

70. A device as claimed in claim 69 wherein the plasticiser is selected from the group consisting of naturally derived oils, synthetic oils and liquid oligomers.

71. A device as claimed in claim 10 wherein the gelatinous elastomeric material is a silicone based gel.

72. A device as claimed in claim 1 wherein the valve is configured to be located externally of an internal cavity proximally of an opening to the internal cavity.

73. A device as claimed in claim I wherein the valve is configured to be located within an internal cavity distally of an opening to the internal cavity.

74. A surgical sealing device comprising a sealing valve for sealing across an opening to an internal cavity:
the sealing valve having a lumen extending therethrough;
the valve being twisted to close the lumen for sealing across the opening;
the valve being movable substantially in a radial direction to enable the lumen to be at least partially opened for passage of an object through the lumen to access the internal cavity, while the valve remains twisted.

75. A surgical sealing device comprising a sealing valve for sealing across an opening to internal cavity;
the sealing valve having a lumen extending therethrough;
the valve being twisted to close the lumen for sealing across the opening;
the lumen being at least partially openable upon engagement of an object with the closed lumen and passage of the object through the lumen to access the internal cavity, while the valve remains twisted.

76. A surgical sealing device comprising a sealing valve for sealing across an opening to an internal cavity;
the sealing valve having a lumen extending therethrough;
the valve being twisted to close the lumen for sealing across the opening;
the valve being configured to enable the lumen to be at least partially opened for passage of an object through the lumen to access the internal cavity, while the valve remains twisted;
the valve being constrained longitudinally.

77. A surgical sealing device comprising a sealing valve for sealing across an opening to an internal cavity;
the sealing valve having a lumen extending therethrough;
a first part of the valve being twistable relative to a second part of the valve between an untwisted configuration, in which the lumen is open, and a twisted configuration to close the lumen for sealing across the opening;
in the twisted configuration, the valve being configured to enable the lumen to be at least partially opened for passage of an object through the lumen to access the internal cavity.

78. A surgical assembly comprising:
a retractor device for retracting the sides of an opening to an internal cavity; and
a surgical sealing device as claimed in claim 1.

79. An assembly as claimed in claim 78 wherein the surgical sealing device is mounted to the retractor device.

80. An assembly as claimed in claim 79 wherein the retractor device has a distal end and a proximal end, and the surgical sealing device is located adjacent to the distal end of the retractor device.

81. An assembly as claimed in claim 79 wherein the retractor device has a distal end and a proximal end, and the surgical sealing device is located adjacent to the proximal end of the retractor device.

82. A method of accessing an internal cavity through an opening to the internal cavity, the method comprising the steps of:
providing a sealing valve, the valve being twisted to form a seal;
arranging the twisted valve to seal across the opening; and
passing an object through the twisted valve, while maintaining the seal, to access the internal cavity.

83. A method as claimed in claim 82 wherein the valve has a lumen extending therethrough, and the valve is twisted into a twisted configuration to close the lumen and form the seal.

84. A method as claimed in claim 83 wherein the lumen is at least partially opened by passing of the object through the twisted valve.

85. A method as claimed in claim 84 wherein the step of passing an object through the valve in the twisted configuration causes a third part of the valve to move relative to a fourth part of the valve to at least partially open the lumen.

86. A method as claimed in claim 85 wherein the third part of the valve is moved substantially radially relative to the fourth part of the valve.

87. A method as claimed in claim 83 wherein the method comprises the step of longitudinally constraining the valve in the twisted configuration.

88. A method as claimed in claim 83 wherein the method comprises the step of twisting the valve from an untwisted configuration, in which the lumen is open, to the twisted configuration.

89. A method as claimed in claim 88 wherein the valve is pre-twisted into the twisted configuration.

90. A method as claimed in claim 89 wherein the valve is supplied in the twisted configuration.

91. A method as claimed in claim 90 wherein the step of twisting the valve is performed during manufacture of the sealing valve.

92. A method as claimed in claim 88 wherein the step of twisting the valve is performed during a surgical procedure.

93. A method as claimed in claim 83 wherein the method comprises the step of hooking the valve in the twisted configuration.

94. A method as claimed in claim 82 wherein the valve is arranged within the opening.

95. A method as claimed in claim 82 wherein the valve is arranged externally of the internal cavity proximally of the opening.

96. A method as claimed in claim 82 wherein the valve is arranged within the internal cavity distally of the opening.

97. A method as claimed in claim 83 wherein the method comprises the step of effecting a seal between a sidewall of the lumen and the object passing through the lumen.

98. A method as claimed in claim 82 wherein the method comprises the step of retracting the sides of the opening to the internal cavity.

99. A method as claimed in claim 82 wherein the method comprises the step of mounting the valve to a retractor device.

100. A method as claimed in claim 99 wherein the method comprises the step of effecting a seal between the valve and the retractor device.

101. A method as claimed in claim 83 wherein the object passed through the lumen comprises a surgeon's hand and/or forearm.

102. A method as claimed in claim 83 wherein the object passed through the opening comprises a surgical instrument.

103. A method of accessing an internal cavity through an opening to the internal cavity, the method comprising the steps of:
providing a sealing valve, having a lumen extending therethrough, the sealing valve being twisted into a twisted configuration to close the lumen closed;
arranging the valve in the twisted configuration to seal across the opening;
passing an object through the valve in the twisted configuration to at least partially open the lumen; and
passing the object through the lumen to access the internal cavity.
